(19) United States
(12) Patent Application Publication
(10) Pub. No.: US 2022/0023314 A1
(43) Pub. Date: Jan. 27, 2022

(54) STABLE FORMULATIONS OF ANESTHETICS AND ASSOCIATED DOSAGE FORMS

(71) Applicant: HALO SCIENCE LLC, Morganville, NJ (US)

(72) Inventors: Xudong YUAN, Morganville, NJ (US); Yu HUI, Winston-Salem, NC (US); Tian ZHANG, Richmond (CA)

(21) Appl. No.: 17/312,898

(22) PCT Filed: Dec. 10, 2019
(86) PCT No.: PCT/US19/65539

§ 371 Date: Jun. 10, 2021

Related U.S. Application Data
(60) Provisional application No. 62/777,755, filed on Dec. 10, 2018.
(60) Provisional application No. 62/777,766, filed on Dec. 11, 2018.

(51) Int. Cl. A61K 31/573; A61K 9/107; A61K 31/575; A61K 31/58; A61K 47/24; A61K 47/10; A61K 47/22; A61K 47/12; A61K 47/18

(52) U.S. Cl. CPC A61K 31/573; A61K 9/107; A61K 31/575; A61K 31/58; A61K 47/18; A61K 9/1075; A61K 47/10; A61K 47/22; A61K 47/12; A61K 47/24

(57) ABSTRACT

Provided herein are stable formulations that deliver one or more neuroactive steroid anesthetic agents in a micellar carrier or self-emulsifying system, which formulations are particularly suitable for use as intravenous anesthetics.

### BACKGROUND

### Technical Field

The present disclosure relates in general to the field of drug delivery systems for neuroactive steroid anesthetic agents. The disclosure additionally relates to dosage forms using stabilized mixed-micelle or self-emulsifying drug delivery systems for neuroactive steroid anesthetic agents.

### Description of the Related Art

Drug delivery systems are used as a medium or carrier for delivering an active pharmaceutical agent (API) to a patient. Desirable drug delivery systems help administer the APIs to the systemic circulation or target sites within a specific time frame. A release profile of active pharmaceutical agents in vivo can be fast, slow, or controlled, depending on the nature of the disease and the need for pharmacological treatment.

Alphaxalone (Alfaxalone or 3-α-hydroxy-5-α-ol-pregnan-11,20-dione) has sedating, anesthetic, anticonvulsant, and neuroprotective properties through modulating GABA A receptors (Child et al., British Journal of Anaesthesia 43:2-13, 1971). As a potent neuroactive steroid anesthetic agent, alphaxalone lacks progestational, estrogenic, mineralocorticoid or thymolytic activity.

Althesin® (Glaxo Laboratories Ltd., Greenford, Middlesex, UK) is an intravenous injectable comprised of alphaxalone and alphadolone in a 3:1 ratio. The anesthetic action of Althesin was attributable to alphaxalone. Althesin enabled rapid onset and offset of anesthetic action, with very few irritating effects on blood vessels, and only minor cardiovascular and respiratory side effects.

Alphaxalone and alphadolone have poor water solubility. To improve the solubility of althesin, a polyethoxylated castor oil excipient, Cremophor EL® (CAS registry 61791-12-6), is typically added into the intravenous injectable. By inducing and maintaining anesthesia, the drug was used in clinical anesthetic practice from 1972 to 1984 in many countries. Althesin was withdrawn from the market as an intravenous anesthetic in humans since 1984. Despite having high therapeutic index, Althesin incurred occasional, unpredictable yet severe anaphylactoid reactions to a (Cremophor EL). However, althesin remains widely used in veterinary medicine.

Di-isopropyl phenol (propofol) is the most popular anesthetic agent in contemporary anesthesia. But there are clinical situations where propofol has limited applications, because propofol may suddenly lower blood pressure, reduce cardiac output and adversely impact respiratory control. As active pharmaceutical agent, propofol can lead to cardiovascular and respiratory depression, a serious clinical adverse reaction that costs patient lives if not remedied immediately. The therapeutic index of propofol is approximately 5, which is extremely low because it means that 5 times of the normal anesthetic dose is fatal.

Furthermore, a lipid emulsion formulation of propofol is susceptible to microbial growth if contaminated and the contaminated propofol have caused clinical instances of inadvertent infections. Pain is another problem caused by a lipid formulation of propofol following or during intravenous injection. Aqueous propofol formulations have resulted in increased injection pain. From a clinical care point of view, the incompatibility of propofol formulation with plastic storage containers and plastic syringes dictate special syringe delivery equipment for intravenous anesthesia and sedation. Due to its lipid formulation, side effects of propofol also include hyperlipidemia and related toxicity when given in a larger dose by infusion.

Because of the limitations faced by propofol and the failures in searching for alternative anesthetic agents, there are renewed interests in reformulating alphaxalone. A notable example is Phaxan (PhaxanCD, PHAX, Chemic Labs, Canton, Mass.), an aqueous solution composed of 10 mg/mL alphaxalone and 13% 7-sulfobutylether β-cyclodextrin (betadex).

In preclinical studies, PHAX has fast onset-offset properties as propofol. Given as intravenous anesthetic, PHAX also incurred less cardiovascular depression than propofol. The Phase 1c clinical study of PHAX looking for equivalent anesthetic dose of PHAX was evaluated for safety, efficacy, and quality of recovery from anesthesia and sedation as compared to propofol (John Monagle et al. Anesthesia Analgesia 121:914-924, 2015). The clinical study results showed that no subject complained of pain on injection with PHAX, while 8 out of the 12 subjects given propofol did. Nine PHAX and eight propofol subjects reached depth of anesthesia, BIS (bispectral index) values of ≤50, with median (interquartile range [IQR]) mg/kg dose=0.5 (0.5-0.6) for PHAX and 2.9 (2.4-3.0) for propofol. The lowest median BIS achieved was 27 to 28 for both PHAX and propofol with no significant differences between them for the time of onset and offset of BIS. The concomitant median changes were ˜11% vs ˜19% for systolic blood pressure and ˜25% vs ˜37% for diastolic blood pressure in PHAX- and propofol-treated subjects, respectively. Nine out of the twelve propofol-treated subjects and none out of twelve PHAX-treated subjects required airway support. For patients reaching an equivalent BIS of ≤50: a Richmond Agitation and Sedation Scale score of 0 was achieved at a median of 5 (IQR, 5-10) and 15 (IQR, 10-20) minutes after PHAX and propofol, respectively; BIS came back to 90 at a mean of 21 (SD, 10.1) and 21 (SD, 9.2) minutes after PHAX and propofol administration, respectively. Therefore, PHAX induced fast-onset, short-duration anesthesia with fast cognitive recovery comparable to propofol, but with fewer occurrence of cardiovascular depression or airway obstruction and no pain on injection.

U.S. Pat. No. 8,975,245B2 discloses possible anesthetic formulations of PHAX. In the disclosure, a host/guest complex formulation was provided comprising a neuroactive steroid anesthetic agent and a cyclodextrin or modified form thereof for use of introducing anesthesia or sedation in mammalian subjects. Because a neuroactive steroid anesthetic agent is sparingly soluble in water, the host/guest complex formulation offered a solution for improving the water solubility of the neuroactive steroid anesthetic agent. A particular cyclodextrin disclosed in the disclosure was a sulfoalkyl ether cyclodextrin such as sulfobutyl ether β-cyclodextrin. This compound could be prepared as described in U.S. Pat. No. 5,376,645A. Another disclosed cyclodextrin is an alkyl ether derivative such as a sulfoalkyl ether-alkyl ether cyclodextrin. Furthermore, the disclosure cites other cyclodextrin derivatives such as methylated, hydroxyalkylated, branched, acylated and anionic forms. The anesthetic formulation of the disclosure provides injectable drug delivery system to mammalian subjects and in particular human subjects. Anesthetic agents disclosed in the disclosure comprise a neuroactive steroids such as alphaxalone, alphadolone, et al.

As demonstrated in the Phase 1c clinical study of PHAX, alphaxalone has the potential for being more efficacious with fewer side effects than propofol. However, as demonstrated in the clinical pharmacology of VFEND® (vorico nazole formulated with sulfobutyl ether β-cyclodextrin) IV injection, in patients with moderate or severe renal impairment (creatinine clearance <50 mL/min), sulfobutyl ether β-cyclodextrin can accumulate over the period of therapy (https://www.rxlist.com/vfend-drug.htm#description). Therefore, oral voriconazole should not be used in the patients with renal insufficiency, unless benefit/risk ratio substantiates the use of intravenous voriconazole. In the case of using intravenous voriconazole, serum creatinine levels need to be closely monitored in the patients with renal impairment. The above clinical pharmacological evidence for VFEND suggested that the use of sulfobutyl ether β-cyclodextrin in patients with renal deficiency is a particular concern.

The permeability of cyclodextrin through biological membranes is limited because of its chemical structure, molecular weight and very low octanol/water partition coefficient. Only the free fraction of drug in equilibrium with the drug-cyclodextrin complexes can readily penetrate the lipophilic membranes. Cyclodextrins generally have no ability to enhance permeability of drugs through biological membranes. In fact, the cyclodextrins can impede drug delivery through lipophilic membrane-controlled barriers (Arun Rasheed et al. Scientia Pharmaceutica. 76:567-598, 2008), because the affinity of cyclodextrin with drug is usually too high to release the drug immediately upon the delivery of drug at the site of action.

Alphaxalone is a positive allosteric modulator of GABAa receptors and at high concentrations; it is a direct agonist of the GABAa receptor. The GABAa receptors are widely distributed in the entire central nervous system (hippocampal pyramidal cells, cerebellar granule cells, thalamus, hippocampus, and hypothalamus etc.). However, the physicochemical properties of cyclodextrin do not allow the excipient to carry alphaxalone across the blood brain barrier and enter central nervous system. Therefore, the fraction of alphaxalone formulated in cyclodextrin or its derivatives that are bioavailable to modulate GABAa receptors is substantially small.

Each milliliter of Althesin solution contains 9 mg of alphaxalone and 3 mg of alphadolone. Alphadolone is only half as potent as the former, but is three times more soluble. The two steroids are prepared in 20% of polyoxyethylated castor oil (Cremophor EL). Considering that the higher doses of the anesthetic triggers an increased incidence of side effects without a corresponding increase in sleeping time, a dosage range of 0.05-0.08 mg/kg was suggested to be adequate (Mark Swerdlow Canadian Anaesthetists' Society Journal, 20: 186-191, 1973). In contrast to the effective dose of PHAX, which is 0.5-0.6 mg/kg as recommended by John Monagle et al. (Anesthesia Analgesia 121:914-924, 2015), the effective dose of Althesin is almost 10 times lower. This observation is consistent with above theoretical projection of cyclodextrin's poor permeability across blood brain barrier into central nervous system and therefore only a small fraction of alphaxalone in PHAX bioavailable to GABAa receptors.

Cremophor EL is a surfactant that forms micelles in aqueous solution when it is above the critical micellar concentration. Despite its hypersensitivity adverse reactions, Cremophor EL is a good encapsulating polymer that may significantly improve the solubility of water-insoluble drugs. Because micelles disintegrate when diluted to below its critical micellar concentration, Cremophor EL formulation can effectively release alphaxalone and make it bioavailable for the uptake by central nervous system. While Cremophor EL is a good solvent for solubilize neuroactive steroid anesthetic agent, such as alphaxalone, it is biological active and its use has caused severe anaphylactoid hypersensitivity reactions, hyperlipidemia, abnormal lipoprotein patterns, aggregation of erythrocytes and peripheral neuropathy.

There is a need, therefore, to develop an alternative suitable formulation which could replace propofol-based intravenous anesthetic or to enable the use of a neuroactive steroid anesthetic agent in subjects that are susceptible to hypersensitivity reactions.

### BRIEF SUMMARY

Provided herein are stable formulations that deliver one or more neuroactive steroid anesthetic agents in a micellar carrier, which formulations are particularly suitable for use as intravenous anesthetics.

It is important for any intravenous anesthetic to rapidly induce sedation and loss of consciousness in a patient as soon as it is given; and to allow the patient to regain awareness as soon as it is halted. Micellar formulations usually disintegrate rapidly in the body and can reach great depth in tissue without delaying the drug release of the active pharmaceutical agent from its micellar structures. However, conventional micellar delivery systems, such as those smaller than 100 nm, tend to be unstable in blood circulation, especially close to/or below its critical micelle concentration.

Certain embodiments thus provide a mixed-micelle delivery system comprising a therapeutically effective amount of one or more neuroactive steroid anesthetic or sedative agents, such as alphaxalone, alphadolone, acebrochol, allopregnanolone, eltanolone (pregnanolone), ganaxolone, hydroxydione, minaxolone, Org20599, Org21465, progesterone metabolites, and tetrahydrodeoxycorticosterone and pharmacologically acceptable derivatives, salts and pro-drug forms thereof, one or more surfactants, one or more stabilizers. The one or more stabilizers, which may also serve as permeability enhancers, stabilize the micellar formulation in the circulation while providing an improved permeability through blood brain barrier to make the neuroactive steroid anesthetic agent bioavailable to GABAa receptors and therefor exert its anesthesia functions.

Other embodiments provide stable formulations capable of self-emulsifying into an emulsion upon contacting an aqueous medium, such as water or body fluid. The self-emulsifying system achieves long term shelf-stability while retaining the fast action of the micellar or mixed-micellar formulations. The self-emulsifying delivery system thus comprises a therapeutically effective amount of a neuroactive steroid anesthetic, such as alphaxalone, alphadolone, acebrochol, allopregnanolone, eltanolone (pregnanolone), ganaxolone, hydroxydione, minaxolone, Org20599, Org21465, progesterone metabolites, tetrahydrodeoxycorticosterone, their various salt forms and derivatives, one or more surfactants; one or more stabilizer, and one or more fatty acids or esters. Optionally, the self-emulsifying formulations may further comprise one or more solid carriers.

### DETAILED DESCRIPTION

Provided herein are stable drug delivery systems for delivering neuroactive steroid anesthetic agents. In particular, the stable delivery systems are mix-micelles or self-emulsifying compositions which are capable of protecting the neuroactive steroid anesthetic agents within the micellar structures (e.g., in blood circulation) and release them rapidly at the target site.

By incorporating the one or more surfactants and stabilizers and permeability enhancers disclosed herein, the anesthetic or sedative formulation of the present disclosure have many advantages over other known anesthetics, including for example: 1) the formulation may reduce incidence of pain on injection because it does not contain irritating excipients and it solubilizes active pharmaceutical agents; 2) the suitable active pharmaceutical agents have a therapeutic index of greater than 5, i.e., larger relative to propofol; 3) the anesthetic induction time and awakening time of the formulation are similar to or faster than propofol or Althesin (alphaxalone and alphadolone); 4) the formulation has lowered cost over other cyclodextrin-based formulations because of the inexpensive nature of the excipients disclosed herein and improved bioavailability; 5) the formulation provides enhanced permeability of blood brain barrier for the active pharmaceutical agents to cross and therefore improves the bioavailability of the agents; 6) the self-emulsifying formulation takes form of solid or semi-solid prior to self-emulsification, allowing longer storage and more facile transportation and handling, as well as less chance of microbial contamination.

Various embodiments according to the present disclosure are thus directed to an anesthetic or sedative composition comprising a neuroactive steroid anesthetic formulated with one or more surfactant(s), or modified form thereof to encapsulate as well as solubilize the neuroactive steroid anesthetic agent, and one or more stabilizers and optionally one or more fatty acid or esters. These components are described in further detail below.

### Neuroactive Steroid Anesthetic

The anesthetic or sedative composition comprising a neuroactive steroid anesthetic. The neuroactive steroid anesthetics are typically highly lipophilic, which benefit from being solubilized and stabilized by micellar structure after delivery. The suitable neuroactive steroid anesthetics include, for example, alphaxalone, alphadolone, acebrochol, allopregnanolone, eltanolone (pregnanolone), ganaxolone, hydroxydione, minaxolone, Org20599 ((2β,3α,5β)-21-chloro-3-hydroxy-2-morpholin-4-ylpregnan-20-one), Org21465 (2β-(2,2-Dimethyl-4-morpholinyl)-3α-hydroxy-11,20-dioxo-5α-pregnan-21-yl methanesulfonate), progesterone metabolites, and tetrahydrodeoxycorticosterone and pharmacologically acceptable derivatives, salts and pro-drug forms thereof, or a combination thereof.

In various embodiments, more than one neuroactive steroid anesthetic may be formulated into a single delivery system. For example, alphaxalone and alphadolone may be combined at a fixed ratio, e.g., 3:1.

### Surfactants

Surfactants are present as emulsifiers that take part in the micellar formation. Surfactants are typically amphiphilic molecules containing both hydrophobic groups (e.g., tails) and hydrophilic groups (e.g., heads). Suitable surfactants may be ionic or non-ionic.

Examples of the surfactants include, without limitation, polyethylene glycol-based surfactants such as ethoxylated esters (e.g., Kolliphor HS) and Vitamin E TPGS, polysorbates (e.g., Tween 20, Tween 80), sorbitans (e.g., Span 20, Span 80), phospholipids, cysteic acid-based surfactants such as N-(all-trans-Retinoyl)-L-cysteic acid, N-(13-cis-Retinoyl)-L-cysteic acid, N-(all-trans-Retinoyl)-L-homocysteic acid, N-(13-cis-Retinoyl)-L-homocysteic acid, N-(all-trans-Retinoyl)-L-cysteinesulfinic acid, N-(13-cis-Retinoyl)-L-cysteinesulfinic acid, and their derivatives.

The surfactants help emulsifying lipids that encapsulate the neuroactive steroid anesthetic agent. The surfactants used in this disclosure also facilitate the penetration of the said neuroactive steroid anesthetic agents to cross the blood brain barrier for reaching GABAa receptors, which are the primary pharmacological targets of neuroactive steroid anesthetic agents.

### Emulsion Stabilizer

The anesthetic or sedative composition further comprises emulsion stabilizers or cosurfactants, including, without limitation, phospholipids such as phosphatidylcholine, lecithin, 1,2-distearoyl-sn-glycero-3-phosphoethanolamine-N-[amino(polyethylene glycol) DSPE-PEG (e.g., DSPE-PEG 2000 or DSPE-PEG 5000), and/or bile acids, tocopherols, their derivatives or their salts. The emulsion stabilizers stabilize the emulsions by aggregating on the surfaces of emulsions (e.g., micellar vesicles) and introduces electrostatic repulsion between the emulsion vesicles. The emulsion stabilizers used in this disclosure also facilitate the penetration of the said neuroactive steroid anesthetic agents to cross the blood brain barrier for reaching GABAa receptors, which are the primary pharmacological targets of neuroactive steroid anesthetic agents.

### Oil-Based Solubilizer

Oil-based solubilizers may be mixtures of fatty acids or esters, which are particularly useful for preparing self-emulsifying formulations, as disclosed herein in further detail below. The fatty acids or esters include, for example, medium chain (C6-C12, or preferably C8-C10) triglycerides or diglycerides (e.g., Labrafac WL1349 or Labrafac PG), labraphil, coconut oil, palm kernel oil, soybean oil, oleic oil, and olive oil thereof. Commercially available lipid excipients such as Capmul INJ MCM and Accon INJ MC8-2 are suitable fatty acids mono-, di- or tri-esters. Some of them are natural ingredients that can be easily degraded and disposed by human body. They function as an oil base or solubilizer that have a great capacity to encapsulate lipophilic drugs such as alphaxalone and make it bioavailable at the site of actions.

### Penetration Enhancers

The penetration enhancers can be used to penetrate the blood brain barriers (BBB) in order to improve the drug permeability and achieve faster and higher drug delivery to the brain. The formulation may further comprise one more penetration enhancer selected from the group consisting of borneol, lecithin, claudin-1, occluding, tricellulin, cereport, TAT, regadenoson, and bsAB.

### Additives

Yet another embodiment of the present disclosure is an anesthetic or sedative composition further comprises a bulk agent such as dibasic calcium phosphonate, lactose, dextrose, fructose, methyl cellulose, HPMC, ethyl cellulose, magnesium stearate, croscarmellose sodium, starch, maltodextrin, cyclodextrin, dextran, and etc. The bulk agents may evenly disperse the pre-dilution formulation to a solid self-emulsifying drug delivery system (S-SEDS) and make it flow freely during packaging and handling. Alternatively, it is sometimes not necessary for the formulation to be treated with bulk agents because the formulation is already in a solid form.

In yet another embodiment of the present disclosure, theanesthetic or sedative composition may further comprises a buffer for maintaining the pH within a range of from about pH 5.5 to pH 8. Alternatively, there might not a need for the formulation to be buffered because the pH of the formulation may be from about pH 3 to about pH 10.

In yet another embodiment of the present disclosure, the anesthetic or sedative composition may further comprise a co-polymer for increasing the viscosity and therefore physical stability of the formulation. Possible examples of co-polymers include but not limited to hydroxyl propyl methyl cellulose (HPMC), polyvinyl pyrollidone (PVP), and carboxymethyl cellulose (CMC) and etc.

### Solvents

One or more solvents may also be present in the stable formulations described herein. The solvents are typically hydrophilic and may be water, alcohol-based solvents such as ethanol, or ether such as 2-(2-ethoxyethoxy)ethanol (Transcutol®) or low molecular weight polyethylene glycol, with average Mn of no more than 8000, and preferably no more than 6000. Commercially available PEG solvents include for example Macrogol® 6000. The hydrophilic solvent may be present as a co-solvent to the oil based solubilizer in self-emulsifying formations.

### Mixed Micelle Formulation

Various embodiments the present disclosure provide mixed-micelle systems for delivering a neuroactive steroid anesthetic. The anesthetic formulation allows for injectable administration to mammalian subjects and in particular human patients with minimal pains experienced at the site of injection.

More specifically, one embodiment provides an anesthetic or sedative composition comprising a neuroactive steroid anesthetic, one or more surfactants and one or more emulsion stabilizers, whereby the neuroactive steroid anesthetic is encapsulated as well as solubilized in micellar vesicles. The mix-micelle formulation may further comprise a hydrophilic solvent such as purified water. ether or ethanol. These components are as described herein.

In various specific embodiments, the mix-micelle system comprises alphaxalone, and one or more surfactants selected from the group consisting of N-(all-trans-Retinoyl)-L-cysteic acid, N-(13-cis-Retinoyl)-L-cysteic acid, N-(all-trans-Retinoyl)-L-homocysteic acid, N-(13-cis-Retinoyl)-L-homocysteic acid, N-(all-trans-Retinoyl)-L-cysteinesulfinic acid, N-(13-cis-Retinoyl)-L-cysteinesulfinic acid, Kolliphor HS, Tween, Span, Vitamin E TPGS surfactant, their esters, derivatives and their salts thereof. The above formulations may further comprises one or more emulsion stabilizer selected from the group consisting of lecithin, DSPE-PEG (e.g., DSPE-PEG 2000 or DSPE-PEG 5000), and/or bile acids, their derivatives and their salts. The above formulation may further comprise one more penetration enhancer selected from the group consisting of borneol, lecithin, claudin-1, occluding, tricellulin, cereport, TAT, regadenoson, and bsAB.

In more specific embodiments, the molar ratio of the neuroactive steroid anesthetic to stabilizer(s) is from about 1:0.01 to about 1:100. More specifically, the molar ratio is about 1:1 to about 1:50; even more specifically, the molar ratio is about 1:1 to about 1:10.

In other embodiments, the molar ratio of the neuroactive steroid anesthetic to the surfactant(s) is from about 1:0.01 to about 1:1000. More specifically, the molar ratio is about 1:1 to about 1:100; or more specifically, the molar ratio is about 1:1 to about 1:20; or more specifically, the molar ratio is about 1:1 to about 1:10.

In other embodiments, the neuroactive steroid anesthetic is present in the formulation in an amount of 0.0001% to 90% of the total weight of the formulation. In more specific embodiments, the neuroactive steroid anesthetic is present in an amount of 0.01% to 10%; or more specifically 0.1% to 10%; or more specifically 0.1% to 1%.

### Self-Emulsifying Formulation

A self-emulsifying formulation of alphaxalone described herein can undergo a spontaneous phase transition in contact with injectable diluent or biological fluids and thereafter self-emulsification. A kinetically and thermodynamically favored phase transition with minimum agitation means that the resulted emulsion can be kept as stable emulsion during storage, allowing the complexed active agent to remain embedded in emulsion vesicles that are dispersed evenly in bulk medium such as phosphate buffered saline or human plasma. Prior to dilution and dispersion, the concentrated alphaxalone formulation can take the form of a solid or semi-solid that enables longer storage, and more facile transportation and handling, as well as less chance of microbial contamination. Self-emulsifying formulation modify the interaction between active agent and biological membranes, which in turn lessens undesirable irritation as seen in other formulations and potentially improves drug bioavailability.

The neuroactive anesthetic formulations are prepared as self-emulsifying systems comprising one or more neuroactive steroid anesthetic agents, mixtures of fatty acids or esters, one or more emulsion stabilizers, and/or one or more surfactants. Within the context of the present disclosure, disclosed neuroactive steroid anesthetic agents include but not limited to alphaxalone, alphadolone, acebrochol, allopregnanolone, eltanolone (pregnanolone), ganaxolone, hydroxydione, minaxolone, Org20599, Org21465, progesterone metabolites, tetrahydrodeoxycorticosterone, their pharmacologically acceptable derivatives, salt or pro-drug forms thereof. And disclosed mixtures of fatty acids or esters include but not limited to labrafac, labraphil, coconut oil, palm kernel oil, soybean oil, and olive oil thereof. The self-emulsifying systems disclosed in this disclosure are stabilized with phospholipids such as lecithin and DSPE-PEG, and/or bile acids, their derivatives and their salts. The stabilizer used in this disclosure also facilitates the penetration of the said neuroactive steroid anesthetic agents to cross the blood brain barrier for reaching GABAa receptors, which are the primary pharmacological targets of neuroactive steroid anesthetic agents. And disclosed surfactants include but not limited to Kolliphor HS, Tween 20, Tween 80, Span 20, or Span 80, Vitamin E TPGS, phospholipids, N-(all-trans-Retinoyl)-L-cysteic acid, N-(13-cis-Retinoyl)-L-cysteic acid, N-(all-trans-Retinoyl)-L-homocysteic acid, N-(13-cis-Retinoyl)-L-homocysteic acid, N-(all-trans-Retinoyl)-L-cysteinesulfinic acid, N-(13-cis-Retinoyl)-L-cysteinesulfinic acid, and their derivatives, thereof to emulsify lipids that encapsulate the neuroactive steroid anesthetic agent.

In more specific embodiments, the molar ratio of the neuroactive steroid anesthetic to the emulsion stabilizer(s) is from about 1:0.01 to about 1:100. More specifically, the molar ratio is about 1:1 to about 1:50; even more specifically, the molar ratio is about 1:1 to about 1:10.

In other embodiments, the molar ratio of the neuroactive steroid anesthetic to the surfactant(s) is from about 1:0.01 to about 1:1000. More specifically, the molar ratio is about 1:1 to about 1:100; or more specifically, the molar ratio is about 1:1 to about 1:20; or more specifically, the molar ratio is about 1:1 to about 1:10.

In other embodiments, the molar ratio of the neuroactive steroid anesthetic to the oil-based solubilizer is from about 1:0.01 to about 1:1000. More specifically, the molar ratio is about 1:1 to about 1:100; or more specifically, the molar ratio is about 1:1 to about 1:20; or more specifically, the molar ratio is about 1:1 to about 1:10.

In other embodiments, the neuroactive steroid anesthetic is present in the formulation in an amount of 0.0001% to 90% of the total weight of the formulation. In more specific embodiments, the neuroactive steroid anesthetic is present in an amount of 0.01% to 10%; or more specifically 0.1% to 10%; or more specifically 0.1% to 1%.

When the solid carrier is present, the self-emulsifying formulation is in a solid form. Typically, the solid carrier may be in an amount (w/w) of 10-50% of the total weight of the formulation. More typically, the solid carrier may be in an amount of 15-30% of the total weight of total weight of the formulation.

### Pharmaceutical Use

The mixed-micelle system and self-emulsifying system may be used in a method for inducing or maintaining an unconscious state in a patient in need thereof, comprising: administering to the patient any of the pharmaceutical formulation described herein.

As used herein the patient may be a human or any other mammalian subjects (e.g., for veterinarian use).

Typically, the formulations may be administered parenteral, e.g., via intravenous or intramuscular routes.

### EXAMPLES

The practice of the present disclosure will employ, unless otherwise indicated, conventional techniques of pharmaceutical formulation, medicinal chemistry, biological testing, and the like, which are within the skill of the art. Such techniques are explained fully in the literature. Preparation of various types of pharmaceutical formulations are described, for example, in Lieberman et al., cited supra; and Gibaldi and Perrier, Pharmacokinetics (Marcel Dekker, 1982), provides a description of the testing procedures useful to evaluate drug delivery systems described and claimed herein.

### Example 1

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| Vitamin E TPGS | 50 mg | Surfactant |
| Lecithin | 30 mg | Stabilizer and Enhancer |
| Purified water | 1 mL | Solvent |

### Example 2

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| DSPE-PEG-2000 | 50 mg | Surfactant |
| Lecithin | 30 mg | Stabilizer and Enhancer |
| Purified water | 1 mL | Solvent |

### Example 3

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| DSPE-PEG-2000 | 30 mg | Surfactant |
| Lecithin | 40 mg | Stabilizer and Enhancer |
| Purified water | 1 mL | Solvent |

### Example 4

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| DSPE-PEG-5000 | 50 mg | Surfactant |
| Lecithin | 30 mg | Stabilizer and Enhancer |
| Purified water | 1 mL | Solvent |

### Example 5

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable about 24 hours.

| Ingredient | Quantity | Function |
|---|---|---|
| Alphaxalone | 2 mg | API |
| Span 80 | 50 mg | Surfactant |
| Bile Salt | 50 mg | Stabilizer and Enhancer |
| Purified water | 1 mL | Solvent |

### Example 6

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| Kolliphor HS-15 | 50 mg | Surfactant |
| Lecithin | 30 mg | Stabilizer and Enhancer |
| Purified water | 1 mL | Solvent |

### Example 7

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system was dried in an oven. The dried mixed-micelle system can be reconstituted with water or buffer to form mixed-micelle in liquid.

| Ingredient | Quantity | Function |
|---|---|---|
| Alphaxalone | 2 mg | API |
| Vitamin E TPGS | 50 mg | Surfactant |
| Lecithin | 30 mg | Stabilizer and Enhancer |
| Ethanol | 0.5 mL | Solvent |

### Example 8

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system was dried in an oven. The dried mixed-micelle system can be reconstituted with water or buffer to form mixed-micelle in liquid.

| Ingredient | Quantity | Function |
|---|---|---|
| Alphaxalone | 2 mg | API |
| DSPE-PEG-2000 | 50 mg | Surfactant |
| Lecithin | 30 mg | Stabilizer and Enhancer |
| Ethanol | 1 mL | Solvent |

### Example 9

A mixed-micelle formulation of alphaxalone was prepared using standard techniques known to those skilled in art. Alphaxalone was weighed and mixed with surfactant, stabilizer, and lactose, thereafter obtained a mixed-micelle drug delivery system after gentle mixing. The formed mixed-micelle system was dried in an oven. The dried mixed-micelle system can be reconstituted with water or buffer to form mixed-micelle in liquid.

| Ingredient | Quantity | Function |
|---|---|---|
| Alphaxalone | 2 mg | API |
| Vitamin E TPGS | 50 mg | Surfactant |
| Lecithin | 30 mg | Stabilizer and Enhancer |
| Lactose | 300 mg | Solid Carrier |
| Ethanol | 0.5 mL | Solvent |

### Example 10

A mixed-micelle formulation of alphaxalone was prepared using standard techniques known to those skilled in art. Alphaxalone was weighed and mixed with surfactant, stabilizer, and lactose, thereafter obtained a mixed-micelle drug delivery system after gentle mixing. The formed mixed-micelle system was dried in an oven. The dried mixed-micelle system can be reconstituted with water or buffer to form mixed-micelle in liquid.

| Ingredient | Quantity | Function |
|---|---|---|
| Alphaxalone | 2 mg | API |
| DSPE-PEG-2000 | 50 mg | Surfactant |
| Lecithin | 30 mg | Stabilizer and Enhancer |
| Lactose | 300 mg | Solid Carrier |
| Ethanol | 1 mL | Solvent |

### Example 11

A mixed-micelle formulation of alphaxalone was prepared using standard techniques known to those skilled in art. Progesterone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Progesterone | 2 mg | API |
| Vitamin E TPGS | 50 mg | Surfactant |
| Lecithin | 30 mg | Stabilizer and Enhancer |
| Purified water | 1 mL | Solvent |

### Example 12

A mixed-micelle formulation of alphaxalone was prepared using standard techniques known to those skilled in art. Progesterone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Progesterone | 2 mg | API |
| DSPE-PEG-2000 | 50 mg | Surfactant |
| Lecithin | 30 mg | Stabilizer and Enhancer |
| Purified water | 1 mL | Solvent |

### Example 13

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| N-(all-trans-Retinoyl)-L-cysteic | 40 mg | Surfactant |
| acid methyl ester sodium salt | | |
| Lecithin | 30 mg | Stabilizer and Enhancer |
| Purified water | 1 mL | Solvent |

### Example 14

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| Soluplus | 40 mg | Surfactant |
| Lecithin | 30 mg | Stabilizer and Enhancer |
| Purified water | 1 mL | Solvent |

### Example 15

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| Vitamin E TPGS | 50 mg | Surfactant |
| HSPC | 30 mg | Stabilizer and Enhancer |
| Purified water | 1 mL | Solvent |

### Example 16

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| DSPE-PEG-2000 | 50 mg | Surfactant |
| Egg phosphatidylcholine | 30 mg | Stabilizer and Enhancer |
| Purified water | 1 mL | Solvent |

### Example 17

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| DSPE-PEG-2000 | 30 mg | Surfactant |
| HSPC | 40 mg | Stabilizer and Enhancer |
| Purified water | 1 mL | Solvent |

### Example 18

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| DSPE-PEG-5000 | 50 mg | Surfactant |
| HSPC | 30 mg | Stabilizer and Enhancer |
| Purified water | 1 mL | Solvent |

### Example 19

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| DSPE-PEG-5000 | 50 mg | Surfactant |
| Egg phosphatidylcholine | 30 mg | Stabilizer and Enhancer |
| Borneol | 5 mg | Penetration Enhancer |
| Purified water | 1 mL | Solvent |

### Example 20

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| DSPE-PEG-5000 | 50 mg | Surfactant |
| Ceramide | 30 mg | Stabilizer and Enhancer |
| Purified water | 1 mL | Solvent |

### Example 21

A mixed-micelle formulation of alphaxalone was prepared using standard techniques known to those skilled in art. Alphaxalone was weighed and mixed with surfactant and stabilizer and thereafter obtained a mixed-micelle drug delivery system after gentle mixing. The formed mixed-micelle system in the container were stable over a week.

| Ingredient | Quantity | Function |
|---|---|---|
| Alphaxalone | 2 mg | API |
| DSPE-PEG-5000 | 50 mg | Surfactant |
| phosphatidylethanolamine | 30 mg | Stabilizer and Enhancer |
| Purified water | 1 mL | Solvent |

### Example 22

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one month.

| Ingredient | Quantity | Function |
|---|---|---|
| Alphaxalone | 2 mg | API |
| Labrafac WL1349 | 18 mg | Solvent |
| DSPE-PEG 2000 | 50 mg | Surfactant |
| Lecithin | 30 mg | Stabilizer and Enhancer |
| Purified water | 1 mL | Co-solvent |

### Example 23

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one month.

| Ingredient | Quantity | Function |
|---|---|---|
| Alphaxalone | 2 mg | API |
| Labrafac PG | 19 mg | Solvent |
| DSPE-PEG 2000 | 50 mg | Surfactant |
| Lecithin | 30 mg | Stabilizer and Enhancer |
| Purified water | 1 mL | Co-solvent |

### Example 24

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one month.

| Ingredient | Quantity | Function |
|---|---|---|
| Alphaxalone | 2 mg | API |
| Transcutol | 25 mg | Solvent |
| DSPE-PEG 5000 | 50 mg | Surfactant |
| Lecithin | 30 mg | Stabilizer and Enhancer |
| Purified water | 1 mL | Co-solvent |

### Example 25

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one month.

| Ingredient | Quantity | Function |
|---|---|---|
| Alphaxalone | 2 mg | API |
| Labrafil M 1944cs | 25 mg | Solvent |
| DSPE-PEG 2000 | 50 mg | Surfactant |
| Lecithin | 30 mg | Stabilizer and Enhancer |
| Purified water | 1 mL | Co-solvent |

### Example 26

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one day.

| Ingredient | Quantity | Function |
|---|---|---|
| Alphaxalone | 2 mg | API |
| Labrafac WL1349 | 18 mg | Solvent |
| Vitamin E TPGS | 50 mg | Surfactant |
| Lecithin | 30 mg | Stabilizer and Enhancer |
| Purified water | 1 mL | Co-solvent |

### Example 27

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one month.

| Ingredient | Quantity | Function |
|---|---|---|
| Alphaxalone | 2 mg | API |
| Labrafac | 18 mg | Solvent |
| DSPE-PEG 2000 | 50 mg | Surfactant |
| Lecithin | 30 mg | Stabilizer and Enhancer |
| Purified water | 1 mL | Co-solvent |

### Example 28

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one week.

| Ingredient | Quantity | Function |
|---|---|---|
| Alphaxalone | 2 mg | API |
| Labrafac WL1349 | 18 mg | Solvent |
| Koliphor HS 15 | 50 mg | Surfactant |
| Lecithin | 30 mg | Stabilizer and Enhancer |
| Purified water | 1 mL | Co-solvent |

### Example 29

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one week.

| Ingredient | Quantity | Function |
|---|---|---|
| Alphaxalone | 2 mg | API |
| Labrafac PG | 19 mg | Solvent |
| Koliphor HS 15 | 50 mg | Surfactant |
| Lecithin | 30 mg | Stabilizer and Enhancer |
| Purified water | 1 mL | Co-solvent |

### Example 30

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one week.

| Ingredient | Quantity | Function |
|---|---|---|
| Alphaxalone | 2 mg | API |
| Transcutol | 25 mg | Solvent |
| Koliphor HS 15 | 50 mg | Surfactant |
| Lecithin | 30 mg | Stabilizer and Enhancer |
| Purified water | 1 mL | Co-solvent |

### Example 31

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one day.

| Ingredient | Quantity | Function |
|---|---|---|
| Alphaxalone | 2 mg | API |
| Labrafac PG | 19 mg | Solvent |
| Span 80 | 50 mg | Surfactant |
| Bile acid salt | 30 mg | Stabilizer and Enhancer |
| Purified water | 1 mL | Co-solvent |

### Example 32

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one week.

| Ingredient | Quantity | Function |
|---|---|---|
| Alphaxalone | 2 mg | API |
| Labrafac WL1349 | 18 mg | Solvent |
| N-(all-trans-Retinoyl)-L-cysteic | 40 mg | Surfactant |
| acid methyl ester sodium salt | | |
| Lecithin | 30 mg | Stabilizer and Enhancer |
| Purified water | 1 mL | Co-solvent |

### Example 33

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer, and solid carrier and dried in an oven thereafter obtained a solid self-emulsifying drug delivery system. The system can be reconstituted with water or buffer to obtain a liquid self-emulsifying drug delivery system. The obtained self-emulsifying preparation was stable for over one month.

| Ingredient | Quantity | Function |
|---|---|---|
| Alphaxalone | 2 mg | API |
| Labrafac WL1349 | 18 mg | Solvent |
| DSPE-PEG 2000 | 50 mg | Surfactant |
| Lecithin | 30 mg | Stabilizer and Enhancer |
| Ethanol | 1 mL | Co-solvent |
| Lactose | 300 mg | Solid carrier |

### Example 34

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one day.

| Ingredient | Quantity | Function |
|---|---|---|
| Alphaxalone | 2 mg | API |
| Labrafac WL1349 | 18 mg | Solvent |
| Soluplus | 50 mg | Surfactant |
| Lecithin | 30 mg | Stabilizer and Enhancer |
| Purified water | 1 mL | Co-solvent |

### Example 35

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one day.

| Ingredient | Quantity | Function |
|---|---|---|
| Alphaxalone | 2 mg | API |
| Mono-di-triglyceride | 18 mg | Solvent |
| Poloxyl 40 hydrogenated castor oil | 40 mg | Surfactant |
| Tocopherol | 30 mg | Stabilizer and Enhancer |
| Purified water | 1 mL | Co-solvent |

### Example 36

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one day.

| Ingredient | Quantity | Function |
|---|---|---|
| Alphaxalone | 2 mg | API |
| Oleic acid | 18 mg | Solvent |
| Polyoxyl 35 Castor Oil | 30 mg | Surfactant |
| Lecithin | 40 mg | Stabilizer and Enhancer |
| Borneol | 5 mg | Penetration Enhancer |
| Purified water | 1 mL | Co-solvent |

### Example 37

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one day.

| Ingredient | Quantity | Function |
|---|---|---|
| Alphaxalone | 2 mg | API |
| PEG-400 | 20 mg | Solvent |
| Propylene Glycol | 10 mg | Co-solvent |
| d-alpha tocopheryl polyethylene | 40 mg | Surfactant |
| glycol 1000 succinate | | |
| Purified water | 1 mL | Co-solvent |

### Example 38

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one day.

| Ingredient | Quantity | Function |
|---|---|---|
| Alphaxalone | 2 mg | API |
| Macrogol 6000 | 15 mg | Solvent |
| Capmul INJ MCM | 10 mg | Cosolvent |
| Accon INJ MC8-2 | 30 mg | Surfactant |
| Lecithin | 40 mg | Stabilizer and Enhancer |
| Purified water | 1 mL | Co-solvent |

### Example 39

A self-emulsifying formulation of neuroactive steroid anesthetic Alphaxalone was prepared using standard techniques known to those skilled in art. Alphaxalone was weighed and mixed with solvent, co-solvent, surfactant, and stabilizer/enhancer and thereafter obtained a self-emulsifying drug delivery system. The obtained self-emulsifying preparation was stable for over one day.

| Ingredient | Quantity | Function |
|---|---|---|
| Alphaxalone | 2 mg | API |
| Captex INJ | 15 mg | Solvent |
| Kolliphor HS15 | 30 mg | Surfactant |
| Lecithin | 40 mg | Stabilizer and Enhancer |
| Purified water | 1 mL | Co-solvent |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Application No. 62/777,755 filed Dec. 10, 2018 and U.S. Provisional Application No. 62/777,766 filed Dec. 11, 2018, which applications are hereby incorporated by reference in their entirety.

1. A pharmaceutical formulation of a self-emulsifying system comprising:
a therapeutically effective amount of an active agent selected from alphaxalone, alphadolone, acebrochol, allopregnanolone, eltanolone (pregnanolone), ganaxolone, hydroxydione, minaxolone, (2β,3α,5β)-21-chloro-3-hydroxy-2-morpholin-4-ylpregnan-20-one, 2β-(2,2-Dimethyl-4-morpholinyl)-3α-hydroxy-11,20-dioxo-5α-pregnan-21-yl methanesulfonate, progesterone metabolites, tetrahydrodeoxycorticosterone, their various salt forms and derivatives;
one or more surfactants;
one or more emulsion stabilizers; and
one or more oil-based solubilizers, wherein the pharmaceutical formulation self-emulsifies into an emulsion upon contacting an aqueous medium.

2. The pharmaceutical formulation of claim 1, wherein the amount of a neuroactive steroid anesthetic is an amount of 0.01-10% of the total weight of the formulation.

3. The pharmaceutical formulation of claim 1, wherein the one or more oil-based solubilizers are fatty acids, fatty acid esters, or combination thereof.

4. The pharmaceutical formulation of claim 3 wherein the fatty acid is coconut oil, palm kernel oil, soybean oil, oleic oil, olive oil or a combination thereof; and the fatty acid esters are medium chain (C6-C12) triglyceride or diglycerides.

5. The pharmaceutical formulation of claim 1, wherein the one or more surfactants are Kolliphor HS, Tween 20, Tween 80, Span 20, Span 80, phospholipids, N-(all-trans-Retinoyl)-L-cysteic acid, N-(13-cis-Retinoyl)-L-cysteic acid, N-(all-trans-Retinoyl)-L-homocysteic acid, N-(13-cis-Retinoyl)-L-homocysteic acid, N-(all-trans-Retinoyl)-L-cysteinesulfinic acid, N-(13-cis-Retinoyl)-L-cysteinesulfinic acid, Vitamin E TPGS, or a combination thereof.

6. The pharmaceutical formulation of claim 1, wherein the one or more emulsion stabilizers are phospholipids, DSPE-PEG, and/or bile acids, their derivatives and their salts or a combination thereof.

7. The pharmaceutical formulation of claim 6 wherein the phospholipid is lecithin or egg phosphatidylcholine.

8. The pharmaceutical formulation of claim 1 further comprising one or more hydrophilic co-solvents selected from water, alcohol, or ether.

9. The pharmaceutical formulation of claim 1 further comprising one or more penetration enhancers selected from borneol, lecithin, claudin-1, occluding, tricellulin, cereport, TAT, regadenoson, and bsAB.

10. The pharmaceutical formulation of claim 1 further comprising a solid carrier selected from the group consisting of dibasic calcium phosphonate, lactose, dextrose, fructose, methyl cellulose, HPMC, ethyl cellulose, magnesium stearate, croscarmellose sodium, starch, maltodextrin, cyclodextrin, dextran, and mixtures thereof.

11. The formulation of claim 9, wherein the solid carrier is present in an amount of 10-50% (w/w) of the total weight of the formulation.

12. A method for inducing or maintaining an unconscious state in a patient in need thereof, comprising: administering to the patient a pharmaceutical formulation of claim 1.

13. A pharmaceutical formulation of a mixed-micelle system comprising:
a therapeutically effective amount of a neuroactive steroid anesthetic or sedative agent, selected from alphaxalone, alphadolone, acebrochol, allopregnanolone, eltanolone (pregnanolone), ganaxolone, hydroxydione, minaxolone, (2β,3α,5β)-21-chloro-3-hydroxy-2-morpholin-4-ylpregnan-20-one, 2β-(2,2-Dimethyl-4-morpholinyl)-3α-hydroxy-11,20-dioxo-5α-pregnan-21-yl methanesulfonate, progesterone metabolites, and tetrahydrodeoxycorticosterone and pharmacologically acceptable derivatives, salts and pro-drug forms thereof,
one or more surfactants,
one or more emulsion stabilizers or permeability enhancers, and
a solvent.

14. The pharmaceutical formulation of claim 12, wherein the amount of anesthetic is in an amount of 0.01-10% of the total weight of the formulation.

15. The pharmaceutical formulation of claim 12, wherein the one or more surfactants are DSPE-PEG2000, DSPE-PEG5000, N-(all-trans-Retinoyl)-L-cysteic acid, N-(13-cis-Retinoyl)-L-cysteic acid, N-(all-trans-Retinoyl)-L-homocysteic acid, N-(13-cis-Retinoyl)-L-homocysteic acid, N-(all-trans-Retinoyl)-L-cysteinesulfinic acid, N-(13-cis-Retinoyl)-L-cysteinesulfinic acid, Kolliphor HS, Tween, Span, Vitamin E, Vitamin E TPGS, Vitamin A, esters or derivatives thereof, or combination thereof.

16. The pharmaceutical formulation of claim 12, wherein the one or more emulsion stabilizers are phospholipids, DSPE-PEG, and/or bile acids, their derivatives and their salts or a combination thereof.

17. The pharmaceutical formulation of claim 15 wherein the phospholipid is lecithin, and DSPE-PEG is DSPE-PEG2000 or DSPE-PEG5000.

18. The pharmaceutical formulation of claim 12 further comprising one or more hydrophilic co-solvents selected from water, alcohol, or ether.

19. The pharmaceutical formulation of claim 12 further comprising one or more penetration enhancers selected from borneol, lecithin, claudin-1, occluding, tricellulin, cereport, TAT, regadenoson, and bsAB.

20. A method for inducing or maintaining an unconscious state in a patient in need thereof, comprising: administering to the patient a pharmaceutical formulation of claim 12.
